(19) United States
(12) Patent Application Publication
(10) Pub. No.: US 2003/0216345 A1
(43) Pub. Date: Nov. 20, 2003

(54) Histone deacetylase inhibitor and use thereof

(75) Inventors: Osamu Nakanishi, Chiba (JP); Takayuki Tatamiya, Chiba (JP)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/438,075

(22) Filed: May 15, 2003

Related U.S. Application Data
(60) Provisional application No. 60/389,282, filed on Jun. 18, 2002.

(30) Foreign Application Priority Data
May 15, 2002 (JP) 2002-140586

(51) Int. Cl. A61K 48/00; C07H 21/04

(52) U.S. Cl. 514/44; 514/12; 536/23.2

(57) ABSTRACT

To provide a novel apoptosis inducer, and a method of screening an apoptosis inducer. For example, an apoptosis inducer comprising inhibiting HDAC6 such as an antisense oligonucleotide to the gene of histone deacetylase 6 (HDAC6), and anti-cancer agent comprising this apoptosis inducer, and the present invention also provides a method of screening an apoptosis inducer that inhibits HDAC6, and more specifically a method of screening an apoptosis inducer said method comprising the steps of (1) determining whether or not a test substance inhibits histone deacetylase 6 (HDAC6) by using deacetylation of an acetylated substance that can be a substrate for histone deacetylase 6 (HDAC6) or decrease in the expression of histone deacetylase 6 (HDAC6) as an index; and (2) confirming, when inhibition is present, whether it induces apoptosis of the cell in vitro and/or in vivo.

### BACKGROUND OF INVENTION

1. Field of Invention

The present invention relates to an apoptosis inducer and a method of screening it. The apoptosis inducer is promising as an anti-cancer agent.

2. Related Art

It has been found that histone deacetylase (HDAC) plays an important role in the regulation of gene expression (Khochbin S., Verdel A., Lemercier C., Seigneurin-Berny D., Functional significance of histone deacetylase diversity, Curr. Opin. Genet. Dev. 2001 April; 11(2) 162-6). For histone deacetylases in human, there are many isozymes, which are classified into class I (HDAC isozymes 1, 2, 3, and 8), class II (HDAC isozymes 4, 5, 6, and 7), and class III (SIRT isozymes 1, 2, 3, 4, 5, 6, and 7). Among them, histone deacetylase 6 (HDAC6) is existed in the cytoplasm unlike the other isozymes that are existed in the nucleus. Thus it has a property different from the other isozymes in that histone cannot be its natural substrate, it is inhibited by trichostatin A, it has a unique structure, and the like.

It is known that microtubules that play important roles in cell division contain α-tubulin as a constitutive protein thereof, and the protein undergoes acetylation when microtubules are stabilized with taxol etc. (Piperno, G., LeDizet, M. & Chang, X. -j. (1987) Microtubules containing acetylated α-tubulin in mammalian cells in culture, J. Cell Biol. 104, 289-302). Thus, it is thought that, in the cytoplasm, there are enzymes (α-tubulin-acetylating enzymes) (α-tubulin acetylase) that add an acetyl group to α-tubulin, and enzymes (α-tubulin-deacetylating enzymes) (α-tubulin deacetylase) that remove an acetyl group added to α-tubulin.

It is known that the acetylation of α-tubulin contributes to stabilizing microtubules (Piperno, G., LeDizet, M. & Chang, X. -j. (1987) Microtubules containing acetylated α-tubulin in mammalian cells in culture, J. Cell Biol. 104, 289-302). However, although histone deacetylase 6 (HDAC6) is a deacetylating enzyme for α-tubulin, it is not known that apoptosis is induced in the cell by inhibiting HDAC6.

### SUMMARY OF INVENTION

The present invention intends to provide an apoptosis inducer that generates an anti-cancer effect by inducing apoptosis, and an anti-cancer agent comprising said apoptosis inducer as an active ingredient, as well as a method of screening an apoptosis inducer.

The present inventors have focused on the facts that among the variety of isozymes of histone deacetylases (HDAC), only histone deacetylase 6 (HDAC6) exist in the cytoplasm and its natural substrate is not histone, and, after intensive and extensive research, have confirmed that HDAC6 is a deacetylating enzyme of α-tubulin. The present inventors have further found that apoptosis can be induced in the cell by inhibiting HDAC6 to enhance the acetylation of α-tubulin thereby stabilizing microtubules, and therefore, have completed the present invention.

Thus the present invention provides an apoptosis inducer comprising a substance that inhibits histone deacetylase 6 (HDAC6). The substance that inhibits histone deacetylase 6 (HDAC6) may be a substance that inhibits histone deacetylase 6 (HDAC6) per se or that suppresses the production (synthesis) of histone deacetylase 6 (HDAC6). As an example of a substance that suppresses the synthesis of histone deacetylase 6, there may be mentioned a nucleic acid or a peptide, and as the nucleic acid there may be mentioned an antisense oligonucleotide or a ribozyme of histone deacetylase 6 (HDAC6).

The present invention also provides a method of screening an apoptosis inducer that inhibits HDAC6, and more specifically a method of screening an apoptosis inducer comprising the steps of:

(1) determining whether or not a test substance inhibits histone deacetylase 6 (HDAC6) by using deacetylation of an acetylated substance that can be a substrate for histone deacetylase 6 (HDAC6) or decrease in the expression of histone deacetylase 6 (HDAC6) as an index; and

(2) further confirming, when inhibition occurs, whether it induces apoptosis of the cell in vitro and/or in vivo.

### BRIEF EXPLANATION OF THE DRAWINGS

FIG. 1 is a graph showing that recombinantly produced HDAC6 according to the present invention has a deacetylating activity, and the activity is inhibited by Trichostatin A.

FIG. 2 is a drawing of Western blot that shows that HDAC6 deacetylates acetylated α-tubulin.

FIG. 3 is a drawing of Western blot that shows that some of the tested HDAC6 antisense oligonucleotides inhibit the expression of HDAC6 and enhance the acetylation of α-tubulin.

FIG. 4 is a drawing of Western blot that shows that HDAC6 antisense oligonucleotide Nos. 4, 5, 6 or 16 inhibits the production of HDAC6 and enhances the acetylation of α-tubulin by changing treatment time.

FIG. 5 is a microgram that shows that the introduction of HDAC6 antisense oligonucleotide No. 4 of the present invention induces the microtubule polymerization in cytoplasm on HeLa cells.

FIG. 6 is a graph showing that HDAC6 antisense oligonucleotides No. 4, 5, or 16 of the present invention induces apoptosis in HeLa cells when judged by Annexin V positivity.

FIG. 7 is a graph showing that HDAC6 antisense oligonucleotides No. 4, 5, or 16 of the present invention induces mitochondrial depolarization in HeLa cells when judged by JC-1 staining.

FIG. 8 is a drawing of Western blot that shows that the HDAC6 antisense oligonucleotide No. 16 of the present invention inhibits the production of HDAC6 and enhances the acetylation of α-tubulin in various human cancer cells.

FIG. 9 is a graph showing that the HDAC6 antisense oligonucleotides No. 16 of the present invention induces mitochondrial depolarization in various human cancer cells when judged by JC-1 staining.

FIG. 10 is a graph showing that the HDAC6 antisense oligonucleotide No. 16 of the present invention induces the cell death in various human cancer cells.

FIG. 11 is a micrograph that shows that the HDAC6 antisense oligonucleotide No. 16 of the present invention stabilizes the microtubules and inhibits disappearance of the microtubules by Colcemid.

FIG. 12 shows the cloning of the full-length HDAC6 cDNA and the construction of a transfer vector for baculovirus.

FIG. 13 is continued from FIG. 12.

FIG. 14 shows the construction of a vector for expression of HDAC6 in animal cells.

### EMBODIMENTS FOR CARRYING OUT THE INVENTION

In accordance with the present invention, it was firstly confirmed that HDAC6 is a deacetylating enzyme of α-tubulin. For this purpose, a cDNA encoding HDAC6 was cloned into mammalian expression vector (Example 1), this vector was introduced into a host cell to overexpress HDAC6 in the cell, and the degree of acetylation of α-tubulin was observed. If HDAC6 is a deacetylating enzyme of α-tubulin, the acetyl group of α-tubulin should be removed by HDAC6 overexpress and thereby the amount of acetylated α-tubulin in the cell should be decreased. The nucleotide sequence of cDNA encoding HDAC6 is set forth in SEQ ID NO: 1 and the amino acid sequence encoded thereby is set forth in SEQ ID NO: 2.

As a result, when HDAC6 was overexpress, the degree of acetylation was decreased by 50%, whereas when the vector that does not express HDAC6 (His6-HDAC6(full)/pcDNA3.1(+)AS) or a vector alone was introduced into the cell, the degree of acetylation of α-tubulin was not decreased. As a result, it was demonstrated that HDAC6 is a deacetylating enzyme of α-tubulin.

Then, methods of decreasing the expression of HDAC6 in the cell were investigated. As a method of inhibiting the expression of the HDAC6 gene, by way of example, the inhibition of expression was attempted using a phosphorothioated oligonucleotide complementary to mRNA (antisense oligonucleotide) of HDAC6. As the antisense oligonucleotide, three oligonucleotides that are complementary to sequences comprising the translation initiation codon ATG of HDAC6, i.e. antisense oligonucleotide 1: CATAGTTGAG GAGGCTTGGC (SEQ ID No. 3), antisense oligonucleotide 2: TCCTGGCCGG TTGAGGTCAT (SEQ ID No. 4), and antisense oligonucleotide 3: GGTTGAGGTC ATAGTTGAGG (SEQ ID No. 5); as well as antisense oligonucleotide 4: CCCTGCCGCG GTTCTTCCAC (SEQ ID No. 6) complementary to a sequence upstream of ATG, and antisense oligonucleotide 5: GCTGCCTGGT TGTGGTGGAA (SEQ ID No. 7) complementary to a sequence downstream of ATG were used.

Furthermore, antisense oligonucleotide 6: TTTATTACAT ATGCAACCTT (SEQ ID NO: 8) complementary to an upstream sequence comprising the polyadenylation signal sequence (bases 4082-4087 in SEQ ID NO: 1), and antisense oligonucleotide 7: AACAGCTTGT ACTTTATTAC (SEQ ID NO: 9) complementary to a downstream sequence comprising the polyadenylation signal sequence, antisense oligonucleotide 8: CCTTGACCGT GGTGCACATC (SEQ ID NO: 10) complementary to a downstream sequence comprising a sequence corresponding to the conserved aspartic acid and histidine residues (DH) in the first catalytic domain (nucleotides 352-1305 in SEQ ID NO: 1) and antisense oligonucleotide 9: GTGGTGCACA TCCCAATCTA (SEQ ID NO: 11) complementary to an upstream sequence comprising a sequence corresponding to the above DH, as well as antisense oligonucleotide 10: CCATTACCGT GGTGGACATC (SEQ ID NO: 12) complementary to a downstream sequence comprising a sequence corresponding to the conserved DH in the second catalytic domain (nucleotides 1537-2475 in SEQ ID NO: 1) and antisense oligonucleotide 11: GTGGTGGACA TCCCAATCCA (SEQ ID NO: 13) complementary to an upstream sequence comprising a sequence corresponding to the above DH were used.

Furthermore, in order to cover the entire region of HDAC6 mRNA, antisense oligonucleotide 12: CGCTCCTCAG GGGACTGCCC (SEQ ID NO: 14) complementary to a sequence comprising nucleotides 1-20 of SEQ ID NO: 1, antisense oligonucleotide 13: ACATCAGCTC TTCCTTTTCA (SEQ ID NO: 15) complementary to a sequence comprising nucleotides 501-520, antisense oligonucleotide 14: CCAAGGCACA TTGATGGTAT (SEQ ID NO: 16) complementary to a sequence comprising nucleotides 1001-1020, and antisense oligonucleotide 15: ACTGGCCATG TCAGGATTGG (SEQ ID NO: 17) complementary to a sequence comprising nucleotides 1501-1520, antisense oligonucleotide 16: TCCGTAGGGC ATGCCCACTG (SEQ ID NO: 18) complementary to a sequence comprising nucleotides 2001-2020, antisense oligonucleotide 17: TGGCAGGGTC AGCAGGGGTG (SEQ ID NO: 19) complementary to a sequence comprising nucleotides 2501-2520, antisense oligonucleotide 18: AGCGTGGCTC CCCCAACAGC (SEQ ID NO: 20) complementary to a sequence comprising nucleotides 3001-3020, antisense oligonucleotide 19: AATTCTCTTG GATTGTTCCA (SEQ ID NO: 21) complementary to a sequence comprising nucleotides 3501-3520, and antisense oligonucleotide 20: TATCAGCTCC CTCTTGGGGC (SEQ ID NO: 22) complementary to a sequence comprising nucleotides 4001-4020 were used.

As a result, the effect of suppressing the gene expression, i.e. reducing the production of HDAC6 was exhibited by using antisense oligonucleotides 1 to 5, antisense oligonucleotides 8 to 11, and antisense oligonucleotide 16. Also, enhanced acetylation of α-tubulin was confirmed concomitantly with the decreased production of HDAC6.

Thus, when an antisense oligonucleotide for which the effect of enhancing the acetylation of α-tubulin was confirmed was introduced into cancer cells, it was observed, microtubules stabilized in the cytoplasm and apoptosis were present. Trichostatin A that is known as a HDAC inhibitor was also confirmed to provide the similar effect.

From the above experimental results, it can be reasonably estimated that a substance that inhibits HDAC6 induces apoptosis and is useful as an anti-cancer agent. Thus, by selecting a substance that inhibits the activity of HDAC6 using the deacetylation and/or acetylation of α-tubulin as an index, it is possible to search an apoptosis inducer and an anti-cancer agent.

Since, as substances that inhibit HDAC6, there may be contemplated enzyme inhibitors that inhibit the enzyme HDAC6 and substances that suppress the production (synthesis) of HDAC6, there are methods of searching an enzyme inhibitor that inhibits the enzyme HDAC6 and methods of searching a substance that inhibits the production (synthesis) of HDAC6.

In order to search an enzyme inhibitor of HDAC6, the enzyme HDAC6, an acetylated substance that can be substrate for HDAC 6, and a test substance are reacted, and the deacetylation of the substrate is observed as an index. In the case where the test substance inhibits the deacetylation, the test substance is expected as an apoptosis inducer. Acetylated substances which can be substrates for HDAC6 are, for example, acetylated α-tublin, an acetylated histone, or synthetic peptides in which a lysine residue has been acetylated. As a method for detecting a deacetylation, any method which can be used for detection of deacetylation may be used, and for example, in the case where the acetylated α-tublin is used as the substrate, a decrease of the acetylated α-tublin and/or an increase of released acetate or deacetylated α-tublin are measured. In addition, for screening of enzyme inhibitors, HDAC Fluorescent activity Assay Kit, AK-500 (BIOMOL), HDAC Assay Kit (CycLex) may be used.

Furthermore, in order to search a substance that inhibits the expression of HDAC6, cells such as an animal cell expressesing HDAC6 is cultured in the presence or in the absense of the test substance, and when the amount of protein and/or mRNA of HDAC6 in the cell cultured in the presence of the test substance is decreased compared to that of protein and/or mRNA of HDAC6 in the cell cultured in the absence of the test substance, the test substance is an expression-suppressant, and may be judged to be an apoptosis inducer.

The amount of protein of HDAC6 can be determined by the Western blot method etc., and the amount of mRNA can be determined by the Northern blot method or the RT-PCR method, etc. When the cell is cultured in the presence or in the absence of the test substance, and the ratio of acetylated α-tubulin to α-tubulin in the cell cultured in the presence of the test substance is compared. When the ratio of acetylated α-tubulin in the cell cultured in the presence of the test substance is higher than the ratio of acetylated α-tubulin in the cell cultured in the absence of the test substance, the test substance is an expression suppressant of HDAC6, and may be estimated to be an apoptosis inducer.

As the above cells, any cells that express HDAC6 can be used, and include animal cells, preferably cultured human cells, and most preferably cancer cells, and there may be mentioned HeLa cells, HL-60 cells, A549 cells, HepG2 cells, LoVo cells, SW480 cells, Calu-1 cells, and more preferably HeLa cell, HepG2 cell, LoVo cell, SW480 cell, Calu-1 cell. Also, genetically engineered cells may be used in which DNA encoding cloned HDAC6 has been artificially introduced so as to express HDAC6 in excess.

### EXAMPLES

The present invention will now be explained in more detail with reference to the Examples.

### Example 1

Isolation of Total RNA

A cDNA encoding HDAC6 was cloned by dividing the coding region of HDAC6 into three parts. HL-60 cells (ATCC) were cultured in RPMI 1640 medium containing 10% FCS and were centrifuged at 500×g for 5 minutes to collect the cells. The collected 1×10⁶ cells were lysed with 1 ml of TRIzole reagent (Gibco BRL), to which 200 μl of chloroform was added and suspended, and the aqueous layer was collected. To the collected aqueous layer was added 500 μl of isopropyl alcohol and mixed, which was centrifuged at 12000×g for 10 minutes to precipitate total RNA. The precipitated total RNA was rinsed with 75% ethanol, and was slightly air-dried and then dissolved in 50 μl of water. The concentration of RNA was measured at a wavelength of 260 nm and was stored at −80° C. until use.

Cloning of CDNA encoding HDAC6

The isolated total RNA of HL-60 cells was used for a reverse transcription reaction. For the reaction, the SuperScript II of Gibco BRL was used, and 0.5 μg of Random 9 mers included in TaKaRa RNA PCR Kit (AMV) Ver. 2.1 was used as the primer, and treated as described in the instruction attached to SuperScript II to obtain total cDNA which was used as a template for PCR. For cloning of the upstream (HEAD) region

5′-side (Reverse) primer: CCTCAACTAT GACCTCAACC G (SEQ ID NO: 23)

3′-side (Forward) primer: TGTCCTCCTC CATGTTGTCC (SEQ ID NO: 24) For cloning of the middle stream (MID) region

5′-side (Reverse) primer: GCTGATCCTG TCTCTGGAGG (SEQ ID NO: 25)

3′-side (Forward) primer: TGGTGACCAA CTTAGAACTG G (SEQ ID NO: 26) For cloning of the downstream (TAIL) region

5′-side (Reverse) primer: AAGTTGGTCA CCAAGAAGGC (SEQ ID NO: 27)

3′-side (Forward) primer: AGGCTGGAAT GAGCTACAGC (SEQ ID NO: 28)

With the above total cDNA as template, PCR was performed using the above primer pairs and using LA Taq (Takara) as the polymerase for 30 cycles with each cycle comprising 94° C. for 1 minute, 55° C. for 30 seconds, and 72° C. for 2 minutes. Each cDNA thus amplified was subcloned into a cloning vector pT7blue T-Vector (Novagen) to obtain HDAC6 HEAD/pT7blue, HDAC6 MID/pT7blue, and HDAC6 TAIL/pT7blue, which were then sequenced.

His6 linkers: CTAGATGCCG CGGGGTTCTC ATCATCATCA TCATCA (SEQ ID NO: 29) and TATGATGATG ATGATGATGA GAACCCCGCG GCAT (SEQ ID NO: 30) were phosphorylated with T4 polynucleotide kinase, annealed and then inserted into the XbaI/NdeI sites of the above HDAC6 HEAD/pT7blue, and the vector constructed was digested with XbaI and DraIII to obtain a His6-tagged HEAD (Xba/DraIII) fragment. This His6-tagged HEAD (Xba/DraIII) fragment and a MID (DraIII/Bpu1102I) fragment obtained by digesting the above HDAC6 MID/pT7blue with DraIII and Bpu1102I were inserted into the above HDAC6 TAIL/pT7blue that had been digested with XbaI and DraIII to construct a full-length HDAC6 (His6 fused at N-terminal)/pT7blue (designated as His6-HDAC6(full)/pT7blue).

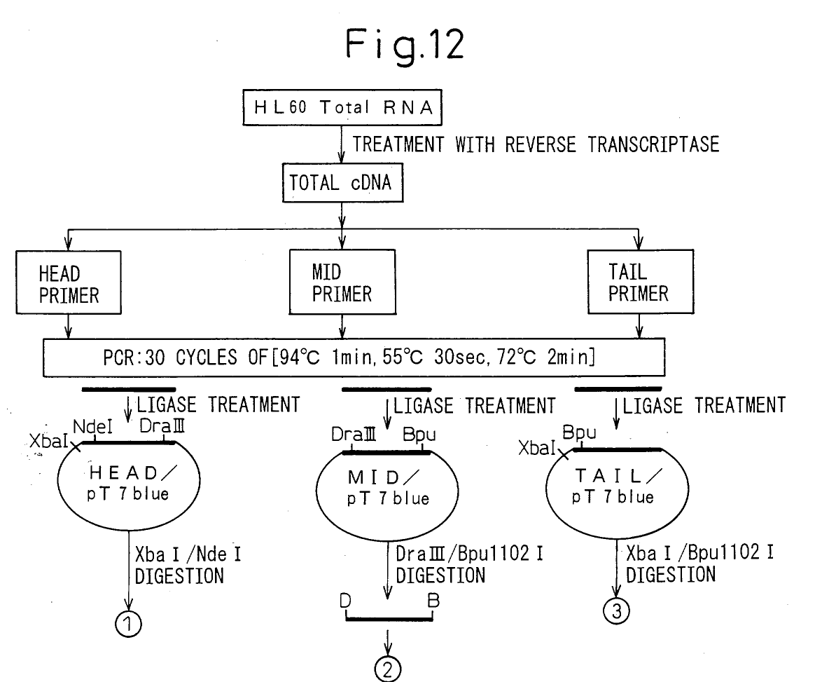

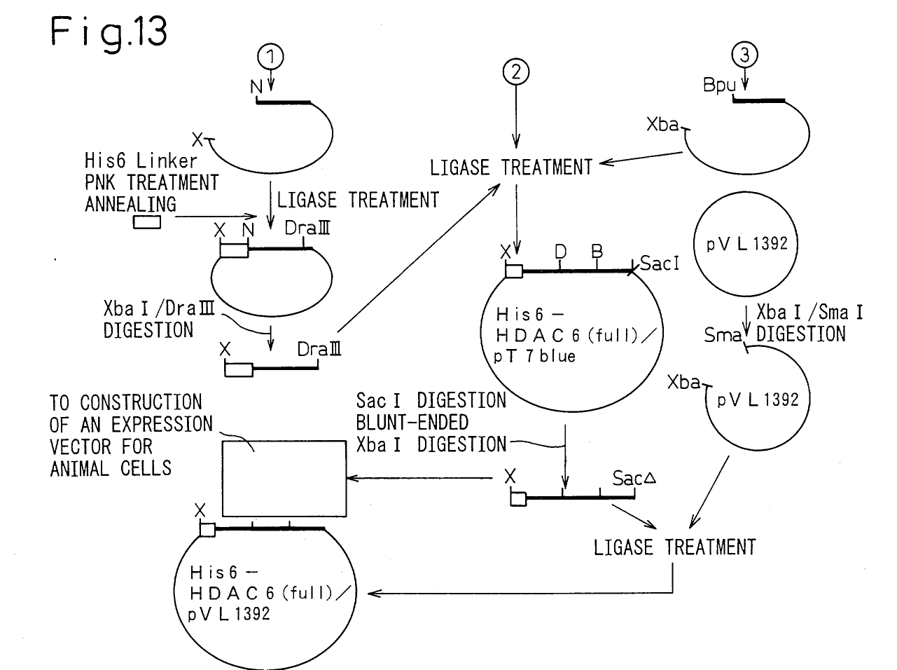

The above His6-HDAC6(full)/pT7blue was digested with SacI, blunt-ended with T4 DNA polymerase, and then digested with XbaI. The fragment obtained was ligated to the XbaI/SmaI site of the vector pVL1392 to construct a transfer vector His6-HDAC6(full)/pVL1392. The above process is described in FIGS. 12 and 13.

Then according to a standard method, this transfer vector was transfected into the Sf-9 cells cultured in the TNM-FH medium. Thus, 4 μg of the above transfer vector His6-HDAC6(full)/pVL1392, 0.5 μg of the Bac-N-Blue linearized AcMNPV DNA (Invitrogen) and 20 l of the InsectinPlus liposome were incubated with 2×10⁶ of Sf-9 cells in one ml of the Grace's insect medium according to the instruction attached to the kit. After cultivating for 7 days, the culture supernatant was collected, subjected to plaque purification, and then the virus was amplified from a single plaque to prepare a high-titer virus stock.

### Example 2

### Production of Recombinant HDAC6 Protein

The virus (about 10⁸ pfu/ml) amplified in Example 1 was diluted 1/100and was infected to 300 ml of Sf-9 cells (106 cells/ml) at a MOI=1, which was then cultured under stirring at 70 rpm at 27° C. for 72 hours. After culturing, the cells were collected by centrifuging at 500×g for 5 minutes and stored at −80° C. The cells were suspended into 6 ml of buffer A (50 mM sodium phosphate buffer, pH 7.5, 300 mM NaCl, 20 mM imidazole) containing protease inhibitors (1 mM PMSF, 2 μM leupeptin, 2 μM pepstatin, 200 nM aprotinin), homogenized at 20 strokes by a Teflon homogenizer, and the homogenate was centrifuged at 20000×g at 4° C. for 30 minutes to obtain a clear cell homogenate. To the cell homogenate obtained, one ml of an adsorbing resin Ni-NTA Superflow (Qiagen) that had been equilibrated with buffer A was added to allow for adsorption at 4° C. for 1 to 2 hours.

The above solution containing the absorbing resin was centrifuged at 2000×g at 4° C. for 5 minutes to remove the supernatant, and the recovered the adsorbing resin was resuspended in 10 ml of buffer A, which is centrifuged at 2000×g at 4° C. for 5 minutes and the supernatant was discarded. The procedure was repeated for three times. The washed resin was suspended in buffer A, and then filled into a column (Econo column, BIO-RAD) and washed with 20 ml of buffer A. The column was eluted with buffer B (50 mM sodium phosphate, pH 7.5, 300 mM NaCl, 300 mM imidazole), and the eluted solution was collected at fractions of 1 ml, which were dialyzed against the dialysis buffer (50 mM Tris-Cl, pH 7.5, 150 mM NaCl, 2 mM EDTA). Protein in the dialysate was determined using Protein Assay kit (BIO-RAD) and stored at −80° C.

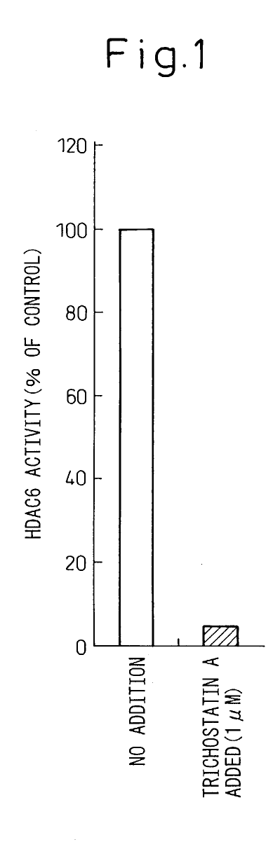

The deacetylating activity of the HDAC6 protein purified as above was determined using HDAC Fluorescent Activity Assay/Drug Discovery Kit (BIOMOL). The determination was also carried out when 1 μM of Trichostatin A, an inhibitor of HDAC6, was presented to the assay system and the result shown in FIG. 1 was obtained. Thus, The deacetylating activity of the test sample was completely inhibited by Trichostatin A, confirming that the recombinantly prepared HDAC6 has the inherent deacetylating activity.

### Example 3

### Construction of an Expression Vector for Forced Expression of HDAC6 and the Promotion of Deacetylation of α-Tubulin by the Forced Expression of HDAC6

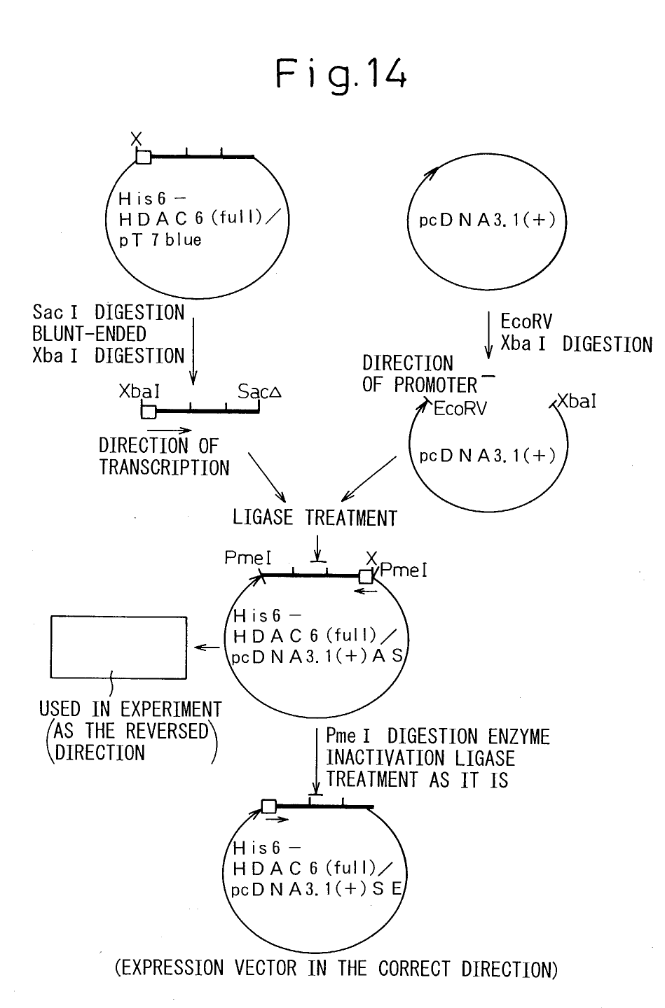

The His6-HDAC6(full)/pT7blue constructed in Example 1 was digested with SacI, blunt-ended with T4 DNA polymerase, and digested with XbaI, and this fragment was inserted into pcDNA3.1(+) (Invitrogen) that had been digested with EcoRV and XbaI to obtain His6-HDAC6(full)/pcDNA3.1(+)AS. Since HDAC6 cDNA was inserted in the reverse direction to the promoter for expression in the constructed vector, said vector was digested with PmeI, and PmeI was inactivated, under which condition pcDNA3.1(+) and HDAC6 fragment were ligated to obtain an expression vector, His6-HDAC6(full)/pcDNA3.1(+)SE, for the forced expression of HDAC6, in which HDAC6 cDNA was inserted in the correct direction to the promoter for expression. The above process is described in FIG. 14.

In a 10-cm tissue culture dish, HeLa cells were plated with the MEM medium containing 10% FCS and 1× nonessential amino acids, 5 μg of His6-HDAC6(full)/pcDNA3.1(+)SE that had been constructed so as to permit the expression of HDAC6 as a histidine-fused protein and 5 μg of pEGFP-C1 (Clontech) were co-transferred using the FuGENE6 transfection reagent (Roche). A similar procedure was followed using His6-HDAC6(full)/pcDNA3.1(+)AS and pcDNA3.1(+). After 48 hours, cells were detached with trypsin and recovered. Using the FACS caliber (Becton Dickinson), only EGFP (Enhanced Green Fluorecence Protein)-positive cells were separately collected.

The recovered cells were lysed with the SDS-PAGE sample buffer (bromophenol blue-free), and protein concentration was determined. After the determination, the cell lysate at a protein amount of 2.5 μg/lane was separated by SDS-PAGE, it was transferred to a PVDF membrane, and was immunoblotted with anti-acetylated α-tubulin antibody (clone 6-11B-1, Sigma), anti-α-tubulin antibody (clone DM1A, Sigma) or anti-RGS His antibody (QIAGEN).

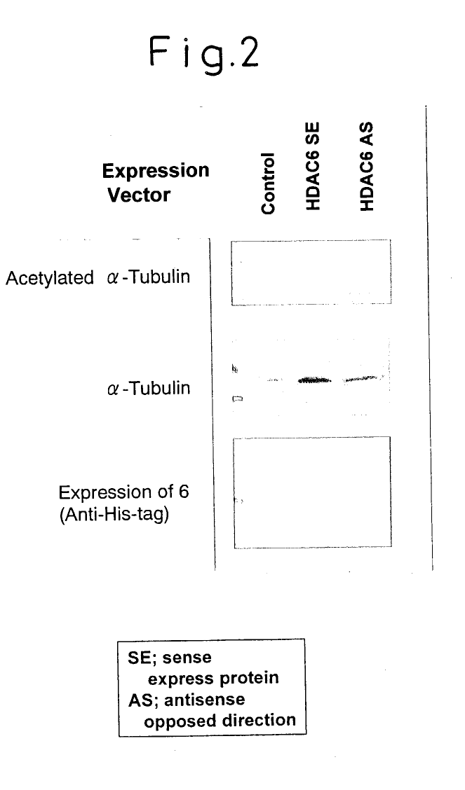

As shown in FIG. 2, there were no changes in the degree of acetylation of α-tubulin in the cells into which His6-HDAC6(full)/pcDNA3.1(+)AS or pcDNA3.1(+), a vector that does not express HDAC6, was introduced, whereas in the cells in which HDAC6 was forcedly expressed the degree of acetylation of α-tubulin was decreased, suggesting that HDAC6 plays a role in the deacetylation of α-tubulin.

### Example 4

### Suppression of HDAC6 Protein Expression Using Phosphorothioated Antisense Oligonucleotide (Antisense Oligonucleotide) and the Result Enhancement of Acetylation of α-Tubulin

Based on the mRNA sequence of the HDAC6 gene (Gene Bank Accession No. NM_{—}006044), 20 different antisense oligonucleotides (SEQ ID NO: 3 to 22) and control oligonucleotide: CCTCTTACCT CAGTTACAAT (SEQ ID NO: 31) (this is derived from a sequence resulting from abnormal splicing at site 705 in pre-mRNA of erythrocyte β globin in hemoglobinopathy thalassanemia, and this oligonucleotide does not have specific targeting sites or biological activity in normal cells) were designed, and were synthesized as phosphorothioated antisense oligonucleotides (Sawady Technology).

The each synthesized antisense oligonucleotide was dissolved in sterile water, and was introduced into HeLa cells using the OligofectAMINE (Invitrogen) according to an attached protocol. The 1.5×10⁵ HeLa cells were plated in a 6-well plate, the final concentration of antisense oligonucleotide was set at 200 nM, and OligofectANINE was used at 3 μl/well.

Forty eight hours after the introduction, the cells were detached with trypsin and collected. After the cells were lysed with a SDS-PAGE sample buffer (BPB-free) and protein concentration was determined, the cell lysate at 25 μg/lane was separated by SDS-PAGE, transferred to a PVDF membrane, and immunoblotted with anti-acetylated α-tubulin antibody (clone 6-11B-1, Sigma), anti-α-tubulin antibody (clone DM1A, Sigma) or anti-HDAC6 antibody (H-300, Santa Cruz).

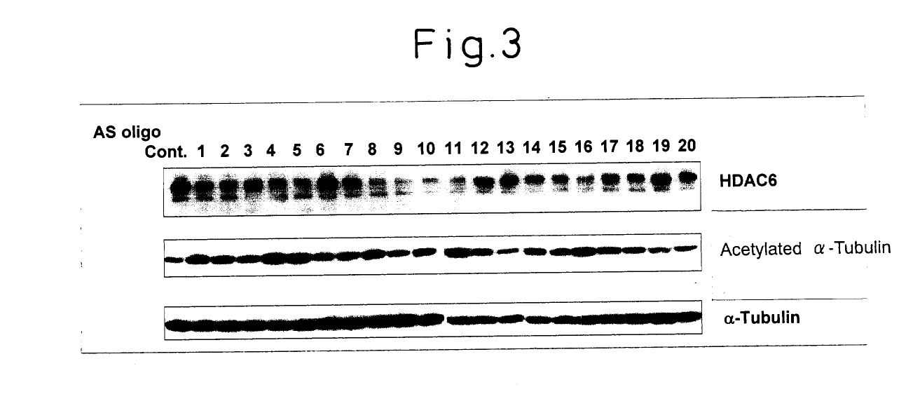

As shown in FIG. 3, about half of the 20 antisense oligonucleotides tested caused the reduction in the amount of expressed HDAC6, confirming that certain antisense oligonucleotides suppress the expression of HDAC6. The control oligo, as expected, did not have any activity of causing the reduction of HDAC6 protein expression.

Also, depending on the reduction in the amount expressed of HDAC6 protein, the acetylation of α-tubulin was increased. This suggested that the deacetylation of α-tubulin is carried out by HDAC6 protein.

Among the above antisense oligonucleotides that exhibited antisense activity, antisense oligonucleotide No. 4 (SEQ ID NO: 6) and No. 5 (SEQ ID NO: 7) in the vicinity of the translation initiation codon ATG, and No. 6 (SEQ ID NO: 8) as the negative control, as well as antisense oligonucleotide No. 16 (SEQ ID NO: 18) located at middle of the coding region and capable of inducing the most potent antisense activity were subjected to a similar experiment as described above by changing the cultivation time (24 hours, 30 hours, 36 hours, and 48 hours) after transfection to cell harvesting.

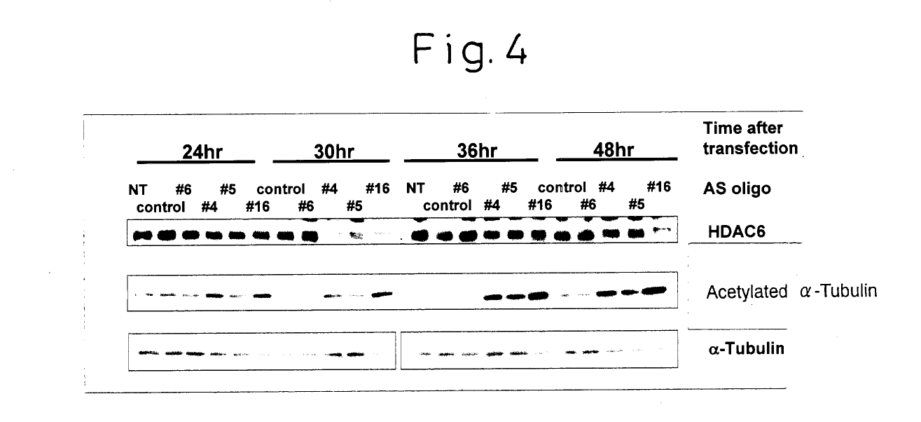

As shown in FIG. 4, it was confirmed that for antisense oligonucleotides Nos. 4, 5 and 16, the acetylation of α-tubulin was enhanced at any time. Accordingly, these three antisense oligonucleotides were used in the subsequent experiments.

### Example 5

### Changes in Microtubule Structure Due to the Reduced Expression of HDAC6 Protein

HeLa Cells were Plated on the Lab-TekII Chambered Coverglass (Nunc), and were Subjected to:

(1) treatment for 16 hours with 1 μM Paclitaxel (Sigma), a microtubule polymerizing agent;

(2) treatment for 16 hours with 1 μM Trichostatin A (Wako), a HDAC inhibitor; or

(3) the introduction of HDAC6 antisense oligonucleotide No. 4 at a final concentration of 200 nM with OligofectAMINE followed by a 24-hour culture.

Then, Lab-TekII Chambered Coverglass was washed with 0.1 M PIPES (pH 6.9), the cells were fixed in 0.5% glutaraldehyde/0.1 M PIPES (pH 6.9) for 10 minutes, washed three times with PBS, permealized three times with 0.5% Triton X100/PBS for 5 minutes each, washed three times with PBS, treated three times with 2.5 mg/ml sodium borohydride/50% ethanol for 10 minutes each, washed three times with PBS, blocked with 10% normal goat serum/PBS, reacted with a primary antibody (a) anti-acetylated α-tubulin antibody (clone 6-11B-1, Sigma) or (b) anti-α-tubulin antibody (clone DM 1A, Sigma)/PBS for 1 hour, washed three times with 0.1% Tween 20/PBS, reacted with a second antibody Alexa Fluor 488-conjugated anti-mouse IgG (Molecular Probe)/PBS for 30 minutes, and washed three times with 0.1% Tween 20/PBS, and then examined under a conforcal laser scanning microscope (Carl Zeiss).

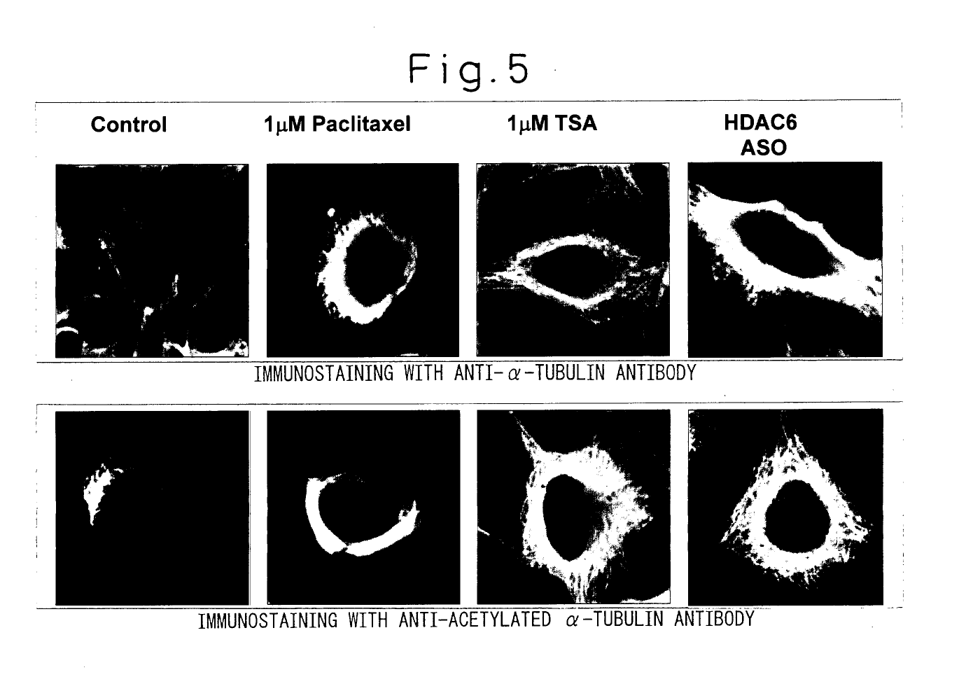

The result is shown in FIG. 5. As compared to the untreated control cells, the cells treated with Paclitaxel, a microtubule polymerization-promoter, when stained with anti-α-tubulin antibody, hyperpolymerized microtubules were observed in the periphery of the nucleus. When stained with anti-acetylated α-tubulin antibody, hyperpolymerized microtubules from the centromere were observed.

In the cells treated with Trichostatin A that inhibits the activity of HDAC6 or a HDAC6 antisense oligonucleotide that suppressed the production of HDAC6, unlike treatment with Paclitaxel that is a polymerization promoter of microtubules, images of polymerized microtubules were observed throughout the cytoplasm. It is suggested that in the cells in which α-tubulin deacetylation was inhibited, microtubule stability was enhanced throughout the cytoplasm by an action mechanism different from when cells were treated with Paclitaxel, a polymerization promoter of microtubules.

### Example 6

### Induction of Apoptosis Due to the Reduced Expression of HDAC6 Protein

HeLa cells (1.5×10⁵/well) were plated into a 6-well plate with MEM medium containing 10% FCS and 1× nonessential amino acids. HDAC6 antisense oligonucleotide No. 4, 5, 6 or 16 at the final concentration of 200 nM or the control oligonucleotide was introduced into the cells with OligofectAMINE at 3 μl/well, and the cells were treated for 24 hours, 30 hours, 36 hours, or 48 hours. The cells were detached by trypsin treatment and collected, and stained with Annexin V-PE (Becton Dickinson) according to a protocol attached, and analyzed using the FACS caliber (Becton Dickinson).

Thus, the collected cells were washed with PBS, suspended in 100 μl of the binding buffer (Becton Dickinson), to which suspension 5 μl of Annexin V-PE was added and incubated for 15 minutes, and the binding buffer was added to make a total volume of 500 μl. This was subjected to FACS analysis.

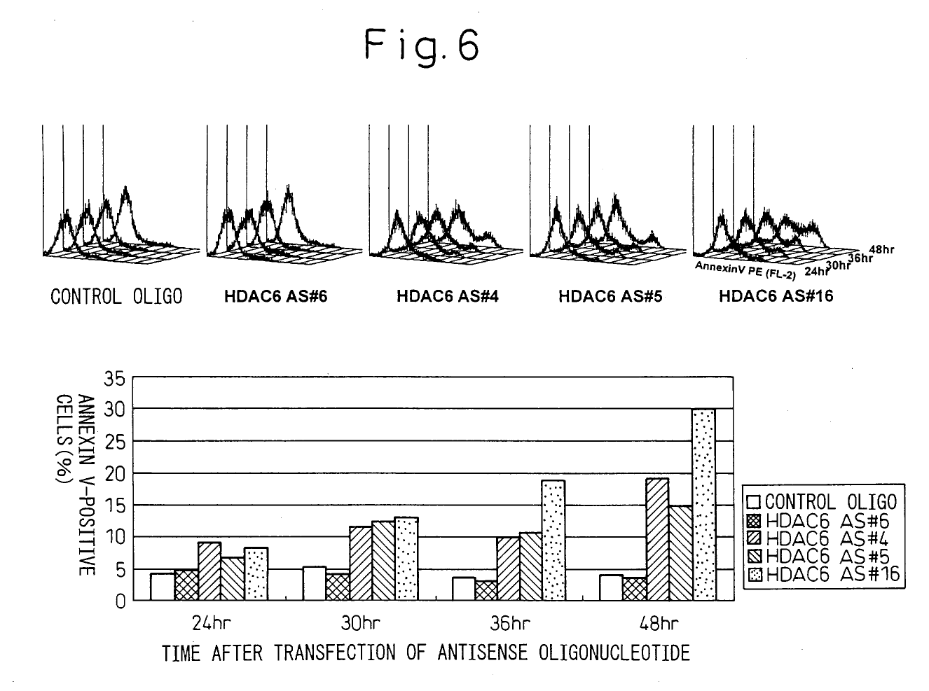

The result is shown in FIG. 6. The ratio of Annexin V-positive cells is increased depending on the reduction of the amount expressed of HDAC6 protein. Annexin V is thought to bind phosphatidylserine on the cell membrane which is extracellularly exposed at an early stage of apoptosis. Thus, it is believed that the inhibition of the function of HDAC6 caused apoptosis in the cell.

### Example 7

### Induction of Depolarization of Mitochondrial Membrane Potential Due to the Reduced Expression of HDAC6 Protein

HeLa cells (1.5×10⁵/well) were plated into a 6-well plate with MEM medium containing 10% FCS and 1× nonessential amino acids. HDAC6 antisense oligonucleotide No. 4, 5 or 16 at the final concentration of 200 nM or the control oligonucleotide was introduced into the cells with OligofectAMINE at 3 μl/well, and the cells were treated for 24 hours, 36 hours or 48 hours. Mitochondrial potential sensor JC-1 (Molecular Probes) was added to the medium at the final concentration of 10 μg/ml and the cells were further caltivated at 37° C. for 10 minutes. Ten minutes later, the cells were detached by trypsin treatment and collected, and analyzed using the FACS caliber (Becton Dickinson).

When Mitochondrial potential sensor JC-1 is added to the medium, it is rapidly incorporated into mitochondria in the normal cells and the cells exhibit red fluorescence (=590 nm; FL-2), whereas in the cells in which mitochondrial membrane potential is depolarized, it is not incorporated into mitochondria and is present in the cytoplasm, and the cells exhibit green fluorescence (=525 nm; FL-1). Therefore, in order to detect the cells in which mitochondrial membrane potential is depolarized, the ratio of green fluorescence-positive cells was graphically shown and used as an index of apoptosis.

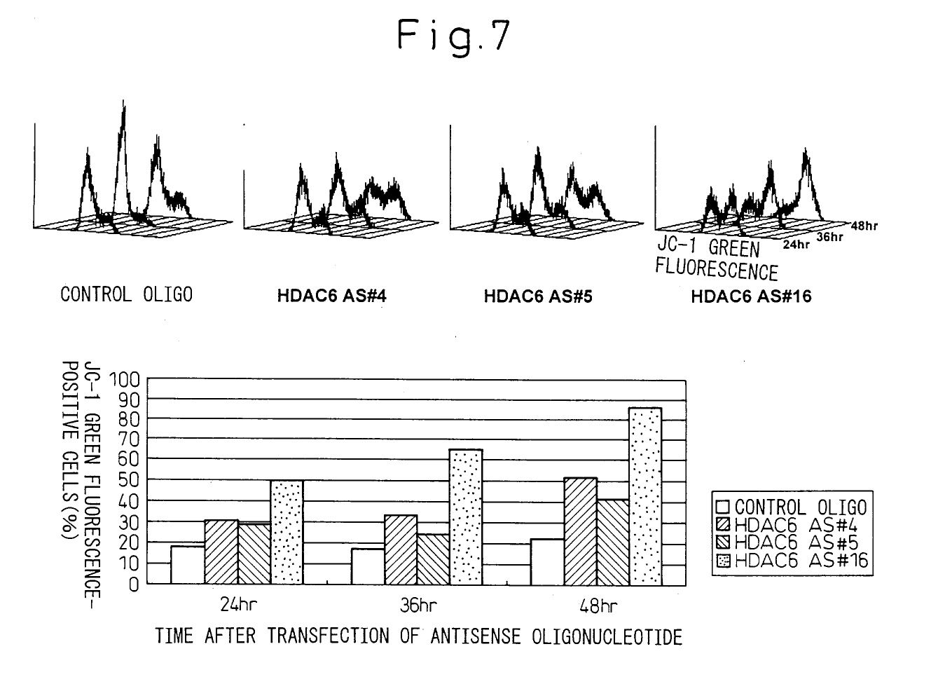

The result is shown in FIG. 7. From the result in FIG. 3, it can be judged that HDAC6 antisense oligonucleotide has an activity of reducing the amount expressed of HDAC6 protein in the order of No. 16, 4 and 5. From the result in FIG. 6, the ratio of cells in which the mitochondrial membrane potential is depolarized is in proportion to the reduction in the amount of expressed HDAC6 protein, and since the inhibition of HDAC6 function causes the enhanced acetylation of α-tubulin in the cell and the depolarization of mitochondrial membrane potential, and therefore it is believed to induce apoptosis.

### Example 8

### Suppression of HDAC6 Protein Expression using Phosphorothioate Antisense Oligonucleotide No. 16 (SEQ ID NO: 18) and the Resulting Enhancement of Acetylation of α-Tubulin

Using an antisense oligonucleotide No. 16 (in this Example, designated as HDAC ASO) and a control oligonucleotide: CCTCTTACCT CAGTTACAAT (SEQ ID NO: 31) (see, Example 4), suppression of HDAC6 protein expression and resulting enhancement of acetylation of α-tubulin by the antisense oligonucleotide was tested in HepG2 cells (Hepatocellular carcinoma), SW480 cells (Colon adenocarcinoma), LoVo cells (Colon adenocarcinoma) and Calu-1 cells (Lung epidermoid carcinoma).

The synthesized antisense oligonucleotide was dissolved in sterile water, and was introduced into the above-mentioned cells using the OligofectAMINE (Invitrogen) according to an attached protocol. The HepG2 cells at an amount of 5×10⁵ cells/well, SW480 cells at an amount of 4×10⁵ cells/well, LoVo cells at an amount of 4×10⁵ cells/well or Calu-1 cells at an amount of 2×10⁵ cells/well were plated in a 6-well plate with RPMI1640 medium (GIBCO) containing 10% FCS, the final concentration of the antisense oligonucleotide was set at 200 nM, and OligofectANINE was used at 3 μl/well.

Forty-eight hours after the introduction of the antisense oligonucleotide, the cells were detached with trypsin and collected. After the cells were lysed with a SDS-PAGE sample buffer (BPB-free) and protein concentration was determined, the cell lysate at 20 μg/lane was separated by SDS-PAGE, transferred to a PVDF membrane, and immunoblotted with anti-acetylated α-tubulin antibody (clone 6-11B-1, Sigma), anti-α-tubulin antibody (clone DM1A, Sigma) or anti-HDAC6 antibody (H-300, Santa Cruz).

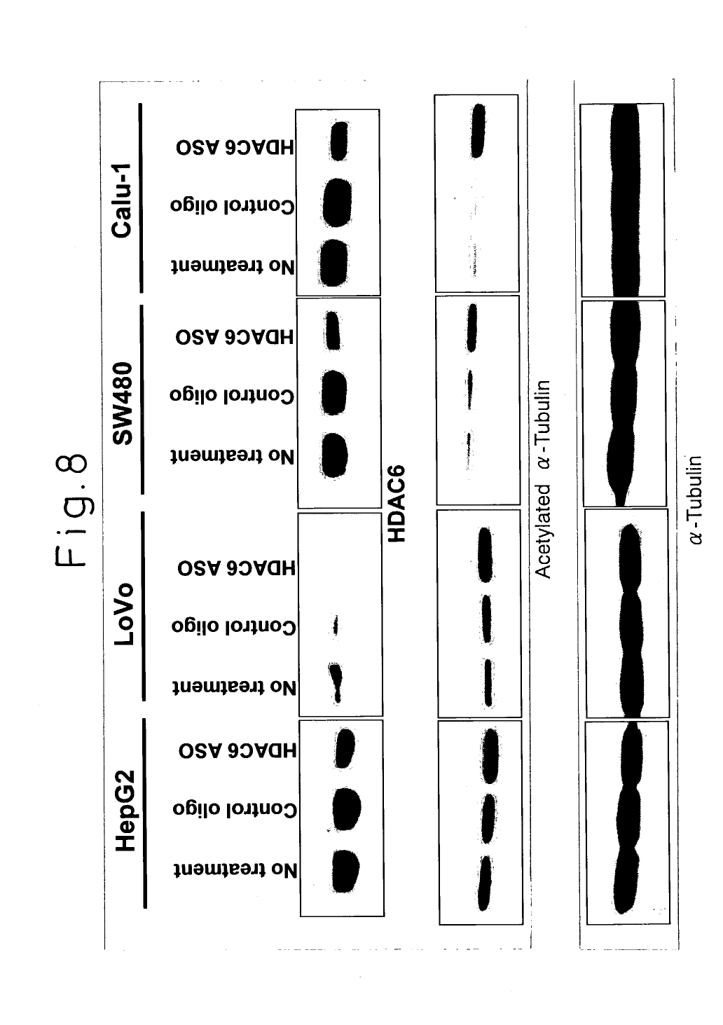

As shown in FIG. 8, it was confirmed that the tested antisense oligonucleotide No. 16 (HDAC6 ASO) caused the reduction in the amount of expressed HDAC6. The control antisense oligonucleotide, as expected, did not have any activity of causing the reduction of HDAC6 protein expression. This confirmed that the deacetylation of α-tubulin is carried out by HDAC6 protein.

### Example 9

### Induction of Depolarization of Mitochondrial Membrane Potential Due to the Reduced Expression of HDAC6 Protein by Antisense Oligonucleotide No. 16

The HepG2 cells at an amount of 5×10⁵ cells/well, SW480 cells at an amount of 4×10⁵ cells/well, LoVo cells at an amount of 4×10⁵ cells/well or Calu-1 cells at an amount of 2×10⁵ cells/well were plated in a 6-well plate with RPMI1640 medium (GIBCO) containing 10% FCS, the HDAC6 antisense oligonucleotide No. 16 or the control anticense oligonucleotide, at the final concentration of 200 nM, was introduced into the cells with OligofectAMINE at 3 μl/well, and the cells were incubated for 48 hours. As a Paclitaxel-treatment control, the cells were incubated with 1 μM Paclitaxel for 24 hours. After the incubations, Mitochondrial potential sensor JC-1 (Molecular Probes) was added to the medium at the final concentration of 10 μg/ml and the cells were further incubated at 37° C. for 10 minutes. Ten minutes later, the cells were detached by trypsin treatment and collected, and analyzed using the FACS caliber (Becton Dickinson).

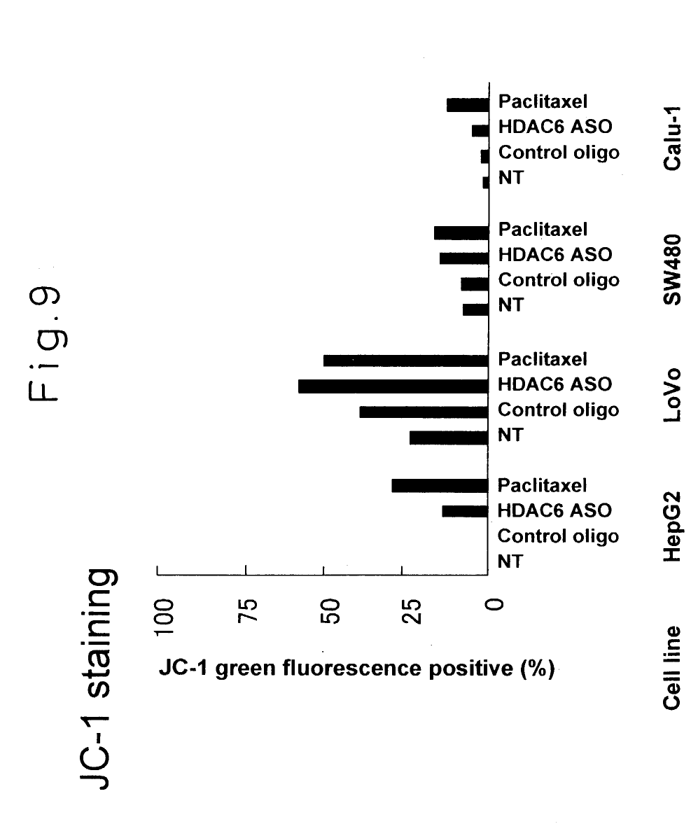

The result is shown in FIG. 9. From the result in FIG. 8, it can be judged that HDAC6 antisense oligonucleotide No. 16 has an activity of reducing the amount expressed of HDAC6 protein. From the result in FIG. 9, the inhibition of HDAC6 function causes the enhanced acetylation of α-tubulin in the cell and the depolarization of mitochondrial membrane potential, and therefore it is believed to induce apoptosis.

### Example 10

### Decrease of the Number of Cells Due to Apoptosis Induced by Antisense Oligonucleotide No. 16

The HepG2 cells at an amount of 5×10⁵ cells/well, SW480 cells at an amount of 4×10⁵ cells/well, LoVo cells at an amount of 4×10⁵ cells/well or Calu-1 cells at an amount of 2×10⁵ cells/well were plated in a 6-well plate with RPMI1640 medium (GIBCO) containing 10% FCS, the HDAC6 antisense oligonucleotide No. 16 or the control anticense oligonucleotide, at the final concentration of 200 nM, was introduced into the cells with OligofectAMINE at 3 μl/well, and the cells were incubated for 48 hours. As a Paclitaxel-treatment control, the cells were incubated with 1 μM Paclitaxel for 24 hours. After the incubations, the cells were collected, and the number of cells was counted by a hemocytometer.

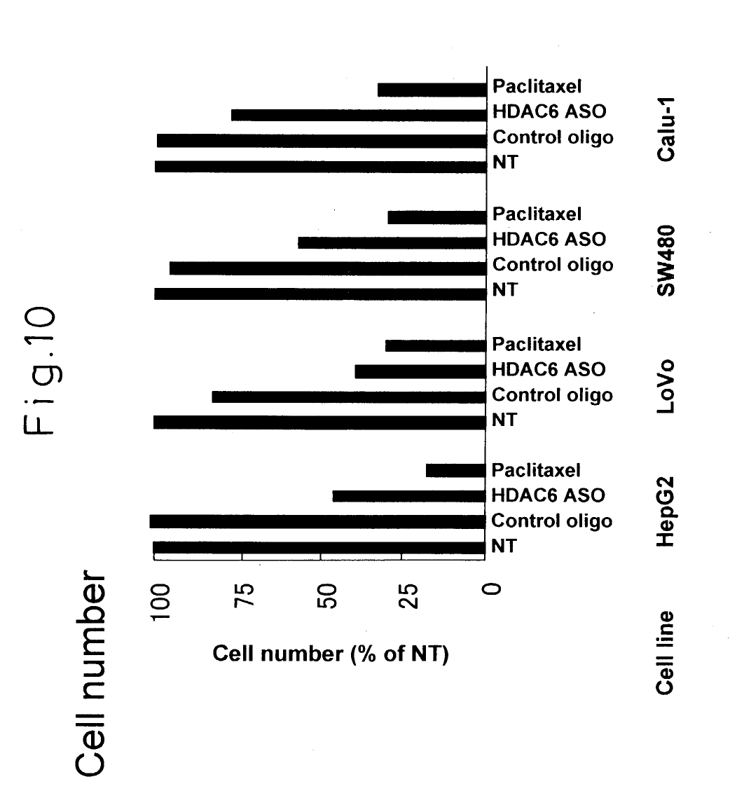

The result is shown in FIG. 10. From the result in FIG. 10, it can be judged that HDAC6 antisense oligonucleotide has an activity of reducing the amount expressed of HDAC6 protein resulting in the induction of the apoptosis.

Results of Examples 8 to 10 suggest that the death of cells is induced by the apoptosis via mitcondria even in cells other than HeLa cells, on the basis of observations that green fluorescence-positive cells are detected and the number of the cells is decreased, both by inhibiting the formation of HDAC6 protein.

### Example 11

### Changes in Microtubule Structure Due to the Expression of HDAC6 Protein Reduced by the Antisense Oligonucleotide No. 16

HeLa cells were plated on the Lab-TekII Chambered Coverglass (Nunc), and were subjected to:

(1) treatment for 16 hours with 1 μM Paclitaxel (Sigma), a microtubule polymerizing agent;

(2) treatment for 16 hours with 1 μM Trichostatin A (Wako), a HDAC inhibitor; or

(3) the introduction of HDAC6 antisense oligonucleotide No. 16 at a final concentration of 200 nM with OligofectAMINE followed by a 24-hour culture.

Colcemid (a derivative of colchicine) was dissolve in ethanol, and added to the medium to the final concentration of 1 μM, and the cells were incubated for 15 minutes (a time in which the microtubule is almost completely disappears without pre-treatment).

Then, Lab-TekII Chambered Coverglass was washed with 0.1 M PIPES (pH 6.9), the cells were fixed in 0.5% glutaraldehyde/0.1 PIPES (pH 6.9) for 10 minutes, washed three times with PBS, permealized three times with 0.5% Triton X100/PBS for 5 minutes each, washed three times with PBS, treated three times with 2.5 mg/ml sodium borohydride/50% ethanol for 10 minutes each, washed three times with PBS, blocked with 10% normal goat serum/PBS, reacted with a primary antibody rat anti-a-tubulin monoclonal antibody (MAB1864; CHEMICON)/PBS for 1 hour, washed three times with 0.1% Tween 20/PBS, reacted with a second antibody Alexa Fluor 488-conjugated anti-rat IgG (Molecular Probe)/PBS for 30 minutes, and washed three times with 0.1% Tween 20/PBS, and then examined under a conforcal laser scanning microscope (Carl Zeiss).

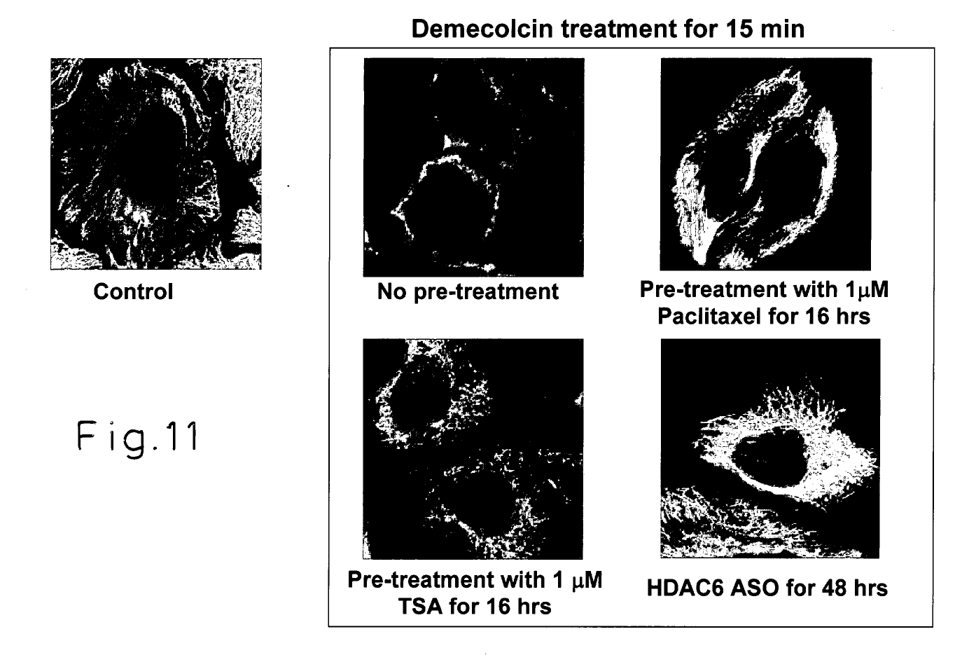

The result is shown in FIG. 11. As compared to the cells not pretreated, and treated with Colcemid, wherein the microtuble disappeared, in the cells pretreated with Paclitaxel that is a polymerization promoter of the microtubules, Tricostatin that is an inhibitor of HDAC6, or the HDAC6 antisense oligonucleotide No. 16, the remaining of the polymerized microtubules was observed. It is suggested that inhibition or repression of HDAC6 promotes acetylation of α-tubulin, and the microtubule was stabilized.

### INDUSTRIAL APPLICABILITY

In accordance with the present invention, it was found that the inhibition of histone deacetylase 6 (HDAC6) causes the enhanced acetylation of α-tubulin, resulting in the induction of apoptosis, and hence a substance that inhibits HDAC6 is promising as an anti-cancer agent. Thus, the present invention provides an apoptosis inducer promising as an anti-cancer agent. The present invention also provides a method of screening an apoptosis inducer promising as an anti-cancer agent.

1. An apoptosis inducer comprising a substance that inhibits histone deacetylase 6 (HDAC6).

2. An apoptosis inducer according to claim 1 wherein said substance that inhibits histone deacetylase 6 (HDAC6) is a nucleic acid or a peptide.

3. An apoptosis inducer according to claim 2 wherein said nucleic acid is an antisense oligonucleotide or a ribozyme of histone deacetylase 6 (HDAC6).

4. An apoptosis inducer according to claim 2 wherein said nucleic acid is an antisense oligonucleotide of histone deacetylase 6 (HDAC6).

5. An apoptosis inducer according to claim 4 wherein said antisense oligonucleotide has a nucleotide sequence complementary to a sequence comprising an initiation codon ATG of the gene encoding histone deacetylase 6 (HDAC6) or a sequence flanking the initiation codon.

6. An apoptosis inducer according to claim 5 wherein said antisense oligonucleotide has a nucleotide sequence CATAGTTGAG GAGGCTTGGC (SEQ ID No. 3), TCCTGGCCGG TTGAGGTCAT (SEQ ID No. 4), GGTTGAGGTC ATAGTTGAGG (SEQ ID No. 5), CCCTGCCGCG GTTCTTCCAC (SEQ ID No. 6), or GCTGCCTGGT TGTGGTGGAA (SEQ ID No. 7).

7. An apoptosis inducer according to claim 4 wherein said antisense oligonucleotide has a nucleotide sequence CCTTGACCGT GGTGCACATC (SEQ ID No. 10), GTGGTGCACA TCCCAATCTA (SEQ ID No. 11), CCATTACCGT GGTGGACATC (SEQ ID No. 12), GTGGTGGACA TCCCAATCCA (SEQ ID No. 13), ACTGGCCATG TCAGGATTGG (SEQ ID No. 17), or TCCGTAGGGC ATGCCCACTG (SEQ ID No. 18).

8. An anti-cancer agent comprising an apoptosis inducer according to any one of claims 1 to 7 as an active ingredient.

9. A method of screening an apoptosis inducer that inhibits histone deacetylase 6 (HDAC6).

10. A method of screening an apoptosis inducer according to claim 9 said method comprising the steps of:
(1) determining whether or not a test substance inhibits histone deacetylase 6 (HDAC6) by using deacetylation of an acetylated substance that can be a substrate for histone deacetylase 6 (HDAC6) or decrease in the expression of histone deacetylase 6 (HDAC6) as an index; and
(2) further confirming, when inhibition is present, whether it induces apoptosis of the cell in vitro and/or in vivo.


SEQUENCE LISTING (31 sequences)

SEQ ID NO: 1 - DNA, 4099 bp, Homo sapiens
  DNA encoding histone deacetylase 6
gggcagtcccctgaggagcggggctggttgaaacgctaggggcgggatctggcggagtggaagaaccgcggcaggggccaagcctcctcaactatgacctcaaccggccaggattccaccacaaccaggcagcgaagaagtaggcagaacccccagtcgccccctcaggactccagtgtcacttcgaagcgaaatattaaaaagggagccgttccccgctctatccccaatctagcggaggtaaagaagaaaggcaaaatgaagaagctcggccaagcaatggaagaagacctaatcgtgggactgcaagggatggatctgaaccttgaggctgaagcactggctggcactggcttggtgttggatgagcagttaaatgaattccattgcctctgggatgacagcttcccggaaggccctgagcggctccatgccatcaaggagcaactgatccaggagggcctcctagatcgctgcgtgtcctttcaggcccggtttgctgaaaaggaagagctgatgttggttcacagcctagaatatattgatctgatggaaacaacccagtacatgaatgagggagaactccgtgtcctagcagacacctacgactcagtttatctgcatccgaactcatactcctgtgcctgcctggcctcaggctctgtcctcaggctggtggatgcggtcctgggggctgagatccggaatggcatggccatcattaggcctcctggacatcacgcccagcacagtcttatggatggctattgcatgttcaaccacgtggctgtggcagcccgctatgctcaacagaaacaccgcatccggagggtccttatcgtagattgggatgtgcaccacggtcaaggaacacagttcaccttcgaccaggaccccagtgtcctctatttctccatccaccgctacgagcagggtaggttctggccccacctgaaggcctctaactggtccaccacaggtttcggccaaggccaaggatataccatcaatgtgccttggaaccaggtggggatgcgggatgctgactacattgctgctttcctgcacgtcctgctgccagtcgccctcgagttccagcctcagctggtcctggtggctgctggatttgatgccctgcaaggggaccccaagggtgagatggccgccactccggcagggttcgcccagctaacccacctgctcatgggtctggcaggaggcaagctgatcctgtctctggagggtggctacaacctccgcgccctggctgaaggcgtcagtgcttcgctccacacccttctgggagacccttgccccatgctggagtcacctggtgccccctgccggagtgcccaggcttcagtttcctgtgctctggaagcccttgagcccttctgggaggttcttgtgagatcaactgagaccgtggagagggacaacatggaggaggacaatgtagaggagagcgaggaggaaggaccctgggagccccctgtgctcccaatcctgacatggccagtgctacagtctcgcacagggctggtctatgaccaaaatatgatgaatcactgcaacttgtgggacagccaccaccctgaggtaccccagcgcatcttgcggatcatgtgccgtctggaggagctgggccttgccgggcgctgcctcaccctgacaccgcgccctgccacagaggctgagctgctcacctgtcacagtgctgagtacgtgggtcatctccgggccacagagaaaatgaaaacccgggagctgcaccgtgagagttccaactttgactccatctatatctgccccagtaccttcgcctgtgcacagcttgccactggcgctgcctgccgcctggtggaggctgtgctctcaggagaggttctgaatggtgctgctgtggtgcgtcccccaggacaccacgcagagcaggatgcagcttgcggtttttgctttttcaactctgtggctgtggctgctcgccatgcccagactatcagtgggcatgccctacggatcctgattgtggattgggatgtccaccacggtaatggaactcagcacatgtttgaggatgaccccagtgtgctatatgtgtccctgcaccgctatgatcatggcaccttcttccccatgggggatgagggtgccagcagccagatcggccgggctgcgggcacaggcttcaccgtcaacgtggcatggaacgggccccgcatgggtgatgctgactacctagctgcctggcatcgcctggtgcttcccattgcctacgagtttaacccagaactggtgctggtctcagctggctttgatgctgcacggggggatccgctggggggctgccaggtgtcacctgagggttatgcccacctcacccacctgctgatgggccttgccagtggccgcattatccttatcctagagggtggctataacctgacatccatctcagagtccatggctgcctgcactcgctccctccttggagacccaccacccctgctgaccctgccacggcccccactatcaggggccctggcctcaatcactgagaccatccaagtccatcgcagatactggcgcagcttacgggtcatgaaggtagaagacagagaaggaccctccagttctaagttggtcaccaagaaggcaccccaaccagccaaacctaggttagctgagcggatgaccacacgagaaaagaaggttctggaagcaggcatggggaaagtcacctcggcatcatttggggaagagtccactccaggccagactaactcagagacagctgtggtggccctcactcaggaccagccctcagaggcagccacagggggagccactctggcccagaccatttctgaggcagccattgggggagccatgctgggccagaccacctcagaggaggctgtcgggggagccactccggaccagaccacctcagaggagactgtgggaggagccattctggaccagaccacctcagaggatgctgttgggggagccacgctgggccagactacctcagaggaggctgtaggaggagctacactggcccagaccacctcggaggcagccatggagggagccacactggaccagactacgtcagaggaggctccagggggcaccgagctgatccaaactcctctagcctcgagcacagaccaccagacccccccaacctcacctgtgcagggaactacaccccagatatctcccagtacactgattgggagtctcaggaccttggagctaggcagcgaatctcagggggcctcagaatctcaggccccaggagaggagaacctactaggagaggcagctggaggtcaggacatggctgattcgatgctgatgcagggatctaggggcctcactgatcaggccatattttatgctgtgacaccactgccctggtgtccccatttggtggcagtatgccccatacctgcagcaggcctagacgtgacccaaccttgtggggactgtggaacaatccaagagaattgggtgtgtctctcttgctatcaggtctactgtggtcgttacatcaatggccacatgctccaacaccatggaaattctggacacccgctggtcctcagctacatcgacctgtcagcctggtgttactactgtcaggcctatgtccaccaccaggctctcctagatgtgaagaacatcgcccaccagaacaagtttggggaggatatgccccacccacactaagccccagaatacggtccctcttcaccttctgaggcccacgatagaccagctgtagctcattccagcctgtaccttggatgaggggtagcctcccactgcatcccatcctgaatatcctttgcaactccccaagagtgcttatttaagtgttaatacttttaagagaactgcgacgattaattgtggatctccccctgcccattgcctgcttgaggggcaccactactccagcccagaaggaaaggggggcagctcagtggccccaagagggagctgatatcatgaggataacattggcgggaggggagttaactggcaggcatggcaaggttgcatatgtaataaagtacaagctgtt

SEQ ID NO: 2 - protein, 1215 aa, Homo sapiens
  Amino acid sequence of histone deacetylase 6
MTSTGQDSTTTRQRRSRQNPQSPPQDSSVTSKRNIKKGAVPRSIPNLAEVKKKGKMKKLGQAMEEDLIVGLQGMDLNLEAEALAGTGLVLDEQLNEFHCLWDDSFPEGPERLHAIKEQLIQEGLLDRCVSFQARFAEKEELMLVHSLEYIDLMETTQYMNEGELRVLADTYDSVYLHPNSYSCACLASGSVLRLVDAVLGAEIRNGMAIIRPPGHHAQHSLMDGYCMFNHVAVAARYAQQKHRIRRVLIVDWDVHHGQGTQFTFDQDPSVLYFSIHRYEQGRFWPHLKASNWSTTGFGQGQGYTINVPWNQVGMRDADYIAAFLHVLLPVALEFQPQLVLVAAGFDALQGDPKGEMAATPAGFAQLTHLLMGLAGGKLILSLEGGYNLRALAEGVSASLHTLLGDPCPMLESPGAPCRSAQASVSCALEALEPFWEVLVRSTETVERDNMEEDNVEESEEEGPWEPPVLPILTWPVLQSRTGLVYDQNMMNHCNLWDSHHPEVPQRILRIMCRLEELGLAGRCLTLTPRPATEAELLTCHSAEYVGHLRATEKMKTRELHRESSNFDSIYICPSTFACAQLATGAACRLVEAVLSGEVLNGAAVVRPPGHHAEQDAACGFCFFNSVAVAARHAQTISGHALRILIVDWDVHHGNGTQHMFEDDPSVLYVSLHRYDHGTFFPMGDEGASSQIGRAAGTGFTVNVAWNGPRMGDADYLAAWHRLVLPIAYEFNPELVLVSAGFDAARGDPLGGCQVSPEGYAHLTHLLMGLASGRIILILEGGYNLTSISESMAACTRSLLGDPPPLLTLPRPPLSGALASITETIQVHRRYWRSLRVMKVEDREGPSSSKLVTKKAPQPAKPRLAERMTTREKKVLEAGMGKVTSASFGEESTPGQTNSETAVVALTQDQPSEAATGGATLAQTISEAAIGGAMLGQTTSEEAVGGATPDQTTSEETVGGAILDQTTSEDAVGGATLGQTTSEEAVGGATLAQTTSEAAMEGATLDQTTSEEAPGGTELIQTPLASSTDHQTPPTSPVQGTTPQISPSTLIGSLRTLELGSESQGASESQAPGEENLLGEAAGGQDMADSMLMQGSRGLTDQAIFYAVTPLPWCPHLVAVCPIPAAGLDVTQPCGDCGTIQENWVCLSCYQVYCGRYINGHMLQHHGNSGHPLVLSYIDLSAWCYYCQAYVHHQALLDVKNIAHQNKFGEDMPHPH

SEQ ID NO: 3 - DNA, 20 bp, Artificial sequence
  Antisense oligonucleotide no.1
catagttgaggaggcttggc

SEQ ID NO: 4 - DNA, 20 bp, Artificial sequence
  Antisense oligonucleotide no.2
tcctggccggttgaggtcat

SEQ ID NO: 5 - DNA, 20 bp, Artificial sequence
  Antisense oligonucleotide no.3
ggttgaggtcatagttgagg

SEQ ID NO: 6 - DNA, 20 bp, Artificial sequence
  Antisense oligonucleotide no.4
ccctgccgcggttcttccac

SEQ ID NO: 7 - DNA, 20 bp, Artificial sequence
  Antisense oligonucleotide no.5
gctgcctggttgtggtggaa

SEQ ID NO: 8 - DNA, 20 bp, Artificial sequence
  Antisense oligonucleotide no.6
tttattacatatgcaacctt

SEQ ID NO: 9 - DNA, 20 bp, Artificial sequence
  Antisense oligonucleotide no.7
aacagcttgtactttattac

SEQ ID NO: 10 - DNA, 20 bp, Artificial sequence
  Antisense oligonucleotide no.8
ccttgaccgtggtgcacatc

SEQ ID NO: 11 - DNA, 20 bp, Artificial sequence
  Antisense oligonucleotide no.9
gtggtgcacatcccaatcta

SEQ ID NO: 12 - DNA, 20 bp, Artificial sequence
  Antisense oligonucleotide no.10
ccattaccgtggtggacatc

SEQ ID NO: 13 - DNA, 20 bp, Artificial sequence
  Antisense oligonucleotide no.11
gtggtggacatcccaatcca

SEQ ID NO: 14 - DNA, 20 bp, Artificial sequence
  Antisense oligonucleotide no.12
cgctcctcaggggactgccc

SEQ ID NO: 15 - DNA, 20 bp, Artificial sequence
  Antisense oligonucleotide no.13
acatcagctcttccttttca

SEQ ID NO: 16 - DNA, 20 bp, Artificial sequence
  Antisense oligonucleotide no.14
ccaaggcacattgatggtat

SEQ ID NO: 17 - DNA, 20 bp, Artificial sequence
  Antisense oligonucleotide no.15
actggccatgtcaggattgg

SEQ ID NO: 18 - DNA, 20 bp, Artificial sequence
  Antisense oligonucleotide no.16
tccgtagggcatgcccactg

SEQ ID NO: 19 - DNA, 20 bp, Artificial sequence
  Antisense oligonucleotide no.17
tggcagggtcagcaggggtg

SEQ ID NO: 20 - DNA, 20 bp, Artificial sequence
  Antisense oligonucleotide no.18
agcgtggctcccccaacagc

SEQ ID NO: 21 - DNA, 20 bp, Artificial sequence
  Antisense oligonucleotide no.19
aattctcttggattgttcca

SEQ ID NO: 22 - DNA, 20 bp, Artificial sequence
  Antisense oligonucleotide no.20
tatcagctccctcttggggc

SEQ ID NO: 23 - DNA, 21 bp, Artificial sequence
  Head reverse primer
cctcaactatgacctcaaccg

SEQ ID NO: 24 - DNA, 20 bp, Artificial sequence
  Head forward primer
tgtcctcctccatgttgtcc

SEQ ID NO: 25 - DNA, 20 bp, Artificial sequence
  Mid reverse primer
gctgatcctgtctctggagg

SEQ ID NO: 26 - DNA, 21 bp, Artificial sequence
  Mid forward primer
tggtgaccaacttagaactgg

SEQ ID NO: 27 - DNA, 20 bp, Artificial sequence
  Tail reverse primer
aagttggtcaccaagaaggc

SEQ ID NO: 28 - DNA, 20 bp, Artificial sequence
  Tail forward primer
aggctggaatgagctacagc

SEQ ID NO: 29 - DNA, 36 bp, Artificial sequence
  His6 linker
ctagatgccgcggggttctcatcatcatcatcatca

SEQ ID NO: 30 - DNA, 34 bp, Artificial sequence
  His6 linker
tatgatgatgatgatgatgagaaccccgcggcat

SEQ ID NO: 31 - DNA, 20 bp, Artificial sequence
  Control oligonucleotide
cctcttacctcagttacaat